(19) United States
(12) Patent Application Publication
(10) Pub. No.: US 2015/0190317 A1
(43) Pub. Date: Jul. 9, 2015

(54) TWO-PHASE COMPOSITION CONTAINING AN ALKYLPOLYGLUCOSIDE AND AN ESTER WITH A MELTING POINT OF LESS THAN 10°C

(71) Applicant: L'OREAL, Paris (FR)

(72) Inventors: Isabelle Bossant, Arcueil (FR); Valérie Gourdin, Chateney Malabry (FR)

(21) Appl. No.: 14/415,363

(22) PCT Filed: Jul. 15, 2013
(86) PCT No.: PCT/IB2013/055818

§ 371 Date: Jan. 16, 2015

Related U.S. Application Data
(60) Provisional application No. 61/675,035, filed on Jul. 24, 2012.

(30) Foreign Application Priority Data
Jul. 16, 2012 (FR) 12 56840

(51) Int. Cl. A61K 8/03; A61Q 1/14; A61K 8/92; A61K 8/37; A61K 8/60; A61K 8/34

(52) U.S. Cl. CPC A61K 8/03; A61K 8/604; A61K 8/342; A61K 8/92; A61K 8/37; A61Q 1/14

(57) ABSTRACT

The present invention relates to a composition for topical application, consisting of a hydrophilic phase and of a separate oily phase, —the hydrophilic phase comprising from 0.05% to 1.5% by weight of at least one non-ethoxylated alkylpolyglucoside relative to the total weight of the hydrophilic phase, and—the oily phase comprising at least 10% by weight, relative to the total weight of the oily phase, of at least one ester with a melting point of less than 0° C. chosen from fatty acid esters with a chain length ranging from 8 to 18 carbon atoms and fatty alcohol esters with a chain length ranging from 8 to 18 carbon atoms, and less than 10% by weight, relative to the total weight of the oily phase, of volatile silicone oil(s).

The present invention relates to a composition for topical application, consisting of two separate phases, a hydrophilic phase and an oily phase, which emulsify readily by shaking to give a composition, and which undergo rapid phase separation after the shaking is stopped.

The present invention also relates to the use of the said composition for removing makeup, for cleansing and/or caring for the skin, the lips and/or the eyes, and/or for caring for the hair.

Compositions of this type consisting of two separate phases, especially a hydrophilic aqueous phase and an oily phase, are generally referred to as “two-phase compositions”. They differ from emulsions in that, when at rest, the two phases are separate instead of being emulsified one in the other. Thus, the two phases are separated at rest by a single interface, whereas, in emulsions, one of the phases is dispersed in the other in the form of a multitude of droplets, and the interfaces are therefore multiple, these interfaces generally being stabilized with emulsifying surfactants and/or emulsifying polymers. The use of two-phase compositions necessitates prior shaking in order to form an extemporaneous emulsion. This emulsion must be of sufficient quality and stability to enable a homogeneous application of the two phases, but such that, when at rest, the two phases become rapidly separated and regain their initial state, this phenomenon being more commonly known as “dephasing”.

Two-phase compositions based on cyclic silicone oils have already been described, for example in documents EP-A-370 856 and EP-A-603 080, especially for removing eye makeup.

Document FR 2 939 662 proposes two-phase compositions based on non-silicone oils, which form, after shaking, a transparent mixture of two immiscible phases and a sharp interface.

In addition, it is increasingly sought to have clear, i.e. transparent, compositions, since, as in the case of water, transparency is a symbol of purity and thus of cleanliness. Transparent compositions are thus particularly appreciated by users, these compositions are generally presented in transparent containers, and the opacity of the two phases is aesthetically detrimental.

The use, in two-phase compositions, of silicone oils, for instance cyclopentasiloxane, in a suitable amount may make it possible to obtain compositions that form, after shaking, a transparent mixture of two immiscible phases.

However, consumers are increasingly in search of formed cosmetic products comprising natural constituents or constituents of natural origin, in particular products not comprising any volatile silicone compounds.

There is thus still a need for a two-phase composition consisting of two separate immiscible phases, which, after shaking, gives an emulsion, while at the same time retaining the properties desired for two-phase compositions, i.e. rapid dephasing into two phases that are preferably transparent, and which is substantially free of volatile silicone compounds.

The Applicant has found, surprisingly, that it is possible to obtain a two-phase composition, which, after shaking, gives an emulsion, and which dephases again rapidly into two phases, having a perfectly sharp interface, without the presence of foam or of residual spirals that are considered unaesthetic and unacceptable to the user, by using a hydrophilic phase comprising one or more non-ethoxylated alkylpolyglucosides in a determined amount and an oily phase comprising one or more particular esters in sufficient amount.

More particularly, a subject of the invention is a composition for topical application, consisting of a hydrophilic phase and of a separate oily phase,
the hydrophilic phase comprising from 0.05% to 1.5% by weight of at least one non-ethoxylated alkylpolyglucoside relative to the total weight of the hydrophilic phase, and
the oily phase comprising at least 10% by weight, relative to the total weight of the oily phase, of at least one ester with a melting point of less than 10° C. chosen from fatty acid esters with a chain length ranging from 8 to 18 carbon atoms and fatty alcohol esters with a chain length ranging from 8 to 18 carbon atoms, and less than 10% by weight, relative to the total weight of the oily phase, of volatile silicone oil(s),
with the exclusion of compositions comprising 75% by weight of hydrophilic phase and 25% by weight of lipophilic phase relative to the weight of the composition, the lipophilic phase containing 45% by weight of isopropyl myristate, relative to the weight of the lipophilic phase, and the hydrophilic phase comprising 0.1% by weight, relative to the weight of the hydrophilic phase, of (85/10/5 C10/12/14)alkylpolyglucoside (1,4) as an aqueous solution containing 55% active material or of (50/50 C8/C10)alkylpolyglucoside as an aqueous solution containing 60% active material.

Since the composition according to the invention is intended for topical application, it contains a physiologically acceptable medium, i.e. a medium that is compatible with the skin, mucous membranes, the hair and the scalp. This composition has makeup-removing qualities.

Advantageously, the hydrophilic and oily phases are transparent.

The composition according to the invention comprises at least one hydrophilic phase and a separate oily phase. These two phases are separate, i.e. they are visible one above the other at rest, and the interface between the two is perfectly sharp. They are preferably transparent at rest, and when the composition is shaken before use, the mixture obtained consists of the emulsion of one phase in the other. The two phases may or may not be coloured.

Advantageously, the two-phase composition gives, after shaking, an emulsion which dephases again into two phases rapidly, i.e. within about one hour.

The word “transparent” means that the composition has a turbidity of less than or equal to 300 NTU. The transparency of a composition may be measured by its turbidity, and the NTU (nephelometric turbidity units) are the units for measuring the turbidity of a composition. The turbidity measurement may be performed, for example, with a model 2100P turbidimeter from the company Hach Compagny, the tubes used for the measurement being referenced AR397A cat 24347-06. The measurements are performed at room temperature (20° C. to 25° C.). The composition of the invention has a turbidity generally ranging from 2 to 300 NTU and preferably from 5 to 200 NTU.

### Hydrophilic Phase

Preferably, the weight ratio between the hydrophilic phase and the oily phase ranges from 90/10 to 10/90, preferably 80/20 to 40/60 and better still from 70/30 to 50/50. The hydrophilic phase thus generally represents from 10% to 90% by weight, preferably from 40% to 80% by weight and better still from 50% to 70% by weight relative to the total weight of the composition.

The hydrophilic phase (also known as the aqueous phase) of the composition according to the invention advantageously comprises water. The water used may be sterile demineralized water and and/or a floral water such as rose water, cornflower water, chamomile water or lime blossom water, and/or a natural spring water or mineral water, for instance: Vittel water, Vichy basin water, Uriage water, Roche Posay water, Bourboule water, Enghien-les-Bains water, Saint Gervais-les-Bains water, Néris-les-Bains water, Allevar-les-Bains water, Digne water, Maizières water, Neyrac-les-Bains water, Lons-le-Saunier water, Eaux Bonnes water, Rochefort water, Saint Christau water, Fumades water, Tercis-les-bains water and Avene water. The hydrophilic phase may also comprise reconstituted spring water, i.e. a water containing trace elements such as zinc, copper, magnesium, etc., reconstituting the characteristics of a spring water.

The hydrophilic phase may also contain any water-soluble or water-dispersible additive. Water-soluble additives that may especially be mentioned are polyols. The term “polyol” should be understood as meaning any organic molecule comprising at least two free hydroxyl groups. Examples of polyols that may be mentioned include glycerol, glycols, for instance butylene glycol, propylene glycol, isoprene glycol, dipropylene glycol, hexylene glycol and polyethylene glycols, sorbitol, sugars such as glucose, and mixtures thereof. According to a preferred embodiment of the invention, the polyol chosen is glycerol, dipropylene glycol or mixtures thereof, or a mixture of glycerol and/or of dipropylene glycol and of one or more other polyols chosen especially from those indicated above: butylene glycol, propylene glycol, isoprene glycol, hexylene glycol, polyethylene glycols, sorbitol, sugars, methylpropanediol and 1,3-propanediol, and mixtures thereof.

The polyols may be present in the composition according to the invention in a content ranging from 0.5% to 60% by weight, preferably from 1% to 50% by weight, better still from 2% to 40% by weight and even better still from 5% to 30% by weight relative to the total weight of the composition.

According to one embodiment, the amount of polyols, in particular of glycerol, is at least 5% by weight relative to the total weight of the composition, preferably at least 10% by weight, better still at least 15% by weight and even better still at least 20% by weight relative to the total weight of the composition.

The hydrophilic phase may also comprise a primary alcohol, i.e. an alcohol comprising from 1 to 6 carbon atoms, such as ethanol and isopropanol. It is preferably ethanol. This alcohol may be present in an amount ranging, for example, from 0.01% to 40% by weight and preferably from 0.1% to 25% by weight relative to the total weight of the composition. The addition of such an alcohol may especially be suitable when the composition according to the invention is used as a product for the body or the hair.

### Alkylpolyglucoside

For the purposes of the present invention, the term “alkylpolyglycoside” means an alkylmonosaccharide (degree of polymerization 1) or an alkylpolysaccharide (degree of polymerization greater than 1).

The alkylpolyglycosides may be used alone or in the form of mixtures of several alkylpolyglycosides. They generally correspond to the following structure:

R(O)(G)ₓ

in which the radical R is a linear or branched C₆-C₃₀ and preferably C₁₂-C₂₂ alkyl radical, G is a saccharide residue and x ranges from 1 to 5, preferably from 1.05 to 2.5 and more preferentially from 1.1 to 2.

The saccharide residue may be chosen from glucose, dextrose, saccharose, fructose, galactose, maltose, maltotriose, lactose, cellobiose, mannose, ribose, dextran, talose, allose, xylose, levoglucan, cellulose and starch. More preferentially, the saccharide residue denotes glucose.

It should also be noted that each unit of the polysaccharide part of the alkylpolyglycoside may be in α or β isomer form, in L or D form, and the configuration of the saccharide residue may be of furanoside or pyranoside type.

It is, of course, possible to use mixtures of alkylpolysaccharides, which may differ from each other in the nature of the borne alkyl unit and/or the nature of the bearing polysaccharide chain.

In particular, mention may be made of alkylpolyglucosides containing an alkyl group comprising from 6 to 30 carbon atoms and preferably from 8 to 16 carbon atoms and containing a glucoside group preferably comprising from 1.2 to 3 saccharide units. Examples that may be mentioned include decylglucoside such as C9/C11-alkyl-polyglucoside (1.4) as an aqueous 40% solution, such as the product sold under the name Mydol 10® by the company Kao Chemicals, 85/10/5 C10/C12/C14-alkyl-polyglucoside (1.4) as an aqueous 55% solution, such as the product sold under the name Oramix NS 10® by the company SEPPIC; and caprylyl/capryl glucoside such as 50/50 C8/C10-alkyl-polyglucoside (2) as an aqueous 60% solution, such as the product sold under the name Oramix CG 110® by the company SEPPIC.

According to a particular embodiment, the alkylpolyglycoside used in a composition according to the invention is chosen from decylglucoside and caprylyl/capryl glucoside.

According to a particular embodiment of the invention, the alkylpolyglycoside may be used as a mixture with at least one fatty alcohol, especially a fatty alcohol containing from 10 to 30 carbon atoms and more particularly from 12 to 22 carbon atoms, as described hereinbelow in the section “co-surfactants”.

In addition, it is particularly advantageous, according to the present invention, to use together a fatty alcohol and an alkylpolyglycoside whose alkyl part is identical to that of the selected fatty alcohol.

Fatty alcohol/alkylpolyglycoside emulsifying mixtures as defined above are known per se. They are described especially in patent applications WO 92/06778, WO 95/13863 and WO 98/47610 and prepared according to the preparation processes indicated in these documents.

Among the fatty alcohol/alkylpolyglycoside mixtures that are particularly preferred, mention may be made of the products sold by the company SEPPIC under the name Montanov®, such as the following mixtures:
cetylstearyl alcohol/cocoyl glucoside—Montanov 82®
arachidyl alcohol and behenyl alcohol/arachidyl glucoside—Montanov 802®
myristyl alcohol/myristyl glucoside—Montanov 14®
cetylstearyl alcohol/cetylstearyl glucoside—Montanov 68®
C₁₄-C₂₂ alcohol/C₁₂-C₂₀ alkyl glucoside—Montanov L®
cocoyl alcohol/cocoyl glucoside—Montanov S®
isostearyl alcohol/isostearyl glucoside—Montanov WO 18®.

According to a particular embodiment, the alkylpolyglycoside used in a composition according to the invention is cetylstearyl glucoside. It is advantageously used in the form of a mixture with cetylstearyl alcohol, also known as cetearyl alcohol.

According to a particular embodiment of the invention, use is thus made of the cetylstearyl alcohol/cetylstearyl glucoside mixture, sold by the company SEPPIC under the name Montanov 68®, consisting of about 20% cetylstearyl glucoside and about 80% cetylstearyl alcohol.

According to a particular embodiment, a composition according to the invention comprises a combination of the monosodium salt of n-stearoyl-L-glutamic acid, more particularly the product sold by the company Ajinomoto under the reference Amisoft HS 11; with a mixture of cetylstearyl alcohol/cetylstearyl glucoside, more particularly the product sold by the company SEPPIC under the name Montanov 68®.

The hydrophilic phase comprises from 0.05% to 1.5% by weight of at least one non-ethoxylated alkylpolyglucoside, preferably from 0.1% to 1% and more preferably from 0.15% to 0.4% relative to the total weight of the hydrophilic phase.

### Oily Phase

The oily phase is also known as the “lipophilic phase”.

The oily phase generally represents from 10% to 90% and preferably from 20% to 60% by weight, and better still from 30% to 50% by weight, relative to the total weight of the composition.

### Volatile Silicone Oils

The oily phase comprises less than 10% by weight of volatile silicone oils relative to the total weight of the oily phase, preferably less than 5%, more preferably less than 3%, better still less than 1% by weight and even better still less than 0.5% by weight of volatile silicone oils. In particular, it is free of volatile silicone oils.

The term “volatile” refers to a compound that can evaporate on contact with the skin in less than one hour, at room temperature and atmospheric pressure. The volatile oil is a volatile cosmetic oil, which is liquid at room temperature, especially having a non-zero vapour pressure, at room temperature and atmospheric pressure, in particular having a vapour pressure ranging from 0.13 Pa to 40 000 Pa (10⁻³ to 300 mmHg), preferably ranging from 1.3 Pa to 13 000 Pa (0.01 to 100 mmHg) and preferentially ranging from 1.3 Pa to 1300 Pa (0.01 to 10 mmHg).

The term “volatile silicone oil” means an oil containing at least one silicon atom, and especially containing Si—O groups.

Examples of volatile silicone oils that may be mentioned include cyclopolydimethylsiloxanes (INCI name: cyclomethicone), such as cyclopentasiloxane, cyclohexasiloxane, octamethylcyclotetrasiloxane, decamethylcyclopentasiloxane, dodecamethylcyclohexasiloxane; linear silicones such as heptamethylhexyltrisiloxane, heptamethyloctyltrisiloxane, hexamethyldisiloxane, octamethyltrisiloxane, decamethyltetrasiloxane and dodecamethylpentasiloxane.

Preferably, the volatile silicone oil is chosen from cyclopentasiloxane and cyclohexasiloxane.

### Non-Volatile Silicone Oils

The oily phase of the composition according to the invention may also comprise at least one oil chosen from silicone oils, such as polymethylsiloxanes, especially PDMS, and phenyl polymethylsiloxanes such as phenyl trimethicones, phenyl dimethicones, phenyltrimethylsiloxydiphenylsiloxanes, diphenyl dimethicones, diphenylmethyldiphenyltrisiloxanes, 2-phenylethyltrimethyl siloxysilicates and polymethylphenylsiloxanes; polysiloxanes modified with fatty acids, fatty alcohols or polyoxyalkylenes, and mixtures thereof.

Preferably, the amount of silicone oil(s) is less than 50% and preferably less than 20% by weight relative to the total weight of the oily phase.

Ester with a Melting Point of Less than 10° C.

The oily phase of the composition according to the invention comprises at least 10% by weight, relative to the total weight of the oily phase, of at least one ester with a melting point of less than 10° C. chosen from fatty acid esters with a chain length ranging from 8 to 18 carbon atoms and fatty alcohol esters with a chain length ranging from 8 to 18 carbon atoms, preferably fatty acid esters with a chain length ranging from 8 to 18 carbon atoms.

These fatty acid esters with a chain length ranging from 8 to 18 carbon atoms may be obtained from a fatty acid with a chain length ranging from 8 to 18 carbon atoms and from a fatty or non-fatty alcohol.

The ester with a melting point of less than 10° C. may be chosen from the oils of formula R1COOR2
in which:
R1 represents a substituted or unsubstituted, linear or branched alkyl or alkoxy radical group comprising from 8 to 18 carbon atoms, preferably from 12 to 18 carbon atoms and better still from 14 to 18 carbon atoms, and possibly comprising one or more ethylenic bonds, and
R2 represents a substituted or unsubstituted, linear or branched alkyl radical comprising from 2 to 20 carbon atoms, preferably from 2 to 18 carbon atoms and better still from 3 to 18 carbon atoms, and possibly comprising one or more ethylenic bonds.

The term “substituted” means that R1 and/or R2 bear one or more substituents chosen, for example, from groups comprising one or more heteroatoms chosen from N, O and S, such as amino, alkoxy or hydroxyl.

In particular, the ester with a melting point of less than 10° C. is an ester of a fatty acid with a chain length ranging from 8 to 18 carbon atoms and of a fatty alcohol with a chain length ranging from 8 to 18 carbon atoms.

Preferably, R1 represents an unsubstituted, saturated, linear or branched alkyl radical comprising from 8 to 18 carbon atoms and better still from 14 to 18 carbon atoms or an unsubstituted, saturated, linear or branched alkoxy radical comprising from 8 to 18 carbon atoms and better still from 8 to 12 carbon atoms. Preferably, R2 represents an unsubstituted, saturated, linear or branched alkyl radical comprising from 2 to 18 carbon atoms and better still from 3 to 18 carbon atoms.

In a non-exhaustive and non-limiting manner, the ester with a melting point of less than 10° C. is chosen from: isocetyl stearate, isopropyl myristate, dicaprylyl carbonate, ethyl hexyl palmitate.

The fatty acid ester oil may be present in a content ranging from 10% to 100% by weight relative to the total weight of the oily phase.

Esters with a Melting Point of Greater than 10° C.

The composition may also comprise at least one fatty acid ester oil with a melting point of greater than 10° C., which may be chosen from the oils of formula R1COOR2

in which:

R1 represents a substituted or unsubstituted, linear or branched alkyl or alkoxy group comprising from 6 to 26 carbon atoms, preferably from 6 to 22 carbon atoms, better still from 8 to 20 carbon atoms and even better still from 8 to 18 carbon atoms, and possibly comprising one or more ethylenic bonds, and
R2 represents a substituted or unsubstituted, linear or branched alkyl radical comprising from 2 to 20 carbon atoms, preferably from 2 to 18 carbon atoms and better still from 3 to 18 carbon atoms, and possibly comprising one or more ethylenic bonds.

The term “optionally substituted” means that R1 and/or R2 may bear one or more substituents chosen, for example, from groups comprising one or more heteroatoms chosen from N, O and S, such as amino, alkoxy or hydroxyl.

Preferably, R1 represents an unsubstituted, saturated, linear or branched alkyl or alkoxy, preferably alkyl, radical comprising from 8 to 20 carbon atoms and better still from 8 to 18 carbon atoms.

Preferably, R2 represents an unsubstituted, saturated, linear or branched alkyl radical comprising from 2 to 18 carbon atoms and better still from 3 to 18 carbon atoms.

Mention may be made especially of isopropyl palmitate.

Preferably, the fatty acid ester oil with a melting point of greater than 10° C. is present in an amount ranging from 30% to 50% by weight relative to the total weight of the oily phase.

### Additional Hydrocarbon-Based Oil

Besides the esters mentioned above, the oily phase of the composition according to the invention may also comprise at least one additional hydrocarbon-based oil chosen from volatile or non-volatile hydrocarbon-based oils.

The term “hydrocarbon-based oil” means an oil formed essentially from, or even consisting of, carbon and hydrogen atoms, and possibly oxygen and nitrogen atoms, and containing no silicon or fluorine atoms; it may contain ester, ether, amine or amide groups.

The content of additional hydrocarbon-based oil(s) may range, for example, from 1% to 60% by weight relative to the total weight of the oily phase.

Examples of hydrocarbon-based oils that may be used in the composition of the invention, and that may be mentioned include:

hydrocarbon-based oils of plant origin, such as perhydrosqualene, liquid triglycerides of fatty acids comprising from 4 to 10 carbon atoms, such as heptanoic or octanoic acid triglycerides, or alternatively, for example, sunflower oil, maize oil, soybean oil, marrow oil, grapeseed oil, sesame seed oil, hazelnut oil, apricot kernel oil, macadamia oil, arara oil, coriander oil, castor oil, avocado oil, caprylic/capric acid triglycerides, such as those sold by the company Stearineries Dubois or those sold under the names Miglyol 810, 812 and 818 by the company Dynamit Nobel, jojoba oil and shea butter oil;

volatile or non-volatile, linear or branched hydrocarbons, of mineral or synthetic origin, and derivatives thereof, such as petroleum jelly oil and hydrogenated polyisobutene such as Parleam® oil; C₈-C₁₆ branched alkanes or isoalkanes (also known as isoparaffins), isododecane, isodecane, isohexadecane, for instance the isoparaffins sold under the trade name Isopar by the company Exxon Chemical or the oils sold under the trade name Permethyl by the company Presperse; and mixtures thereof;

linear alkanes, especially of plant origin, preferably comprising from 7 to 14 carbon atoms;

fatty alcohols that are liquid at room temperature, containing from 8 to 26 carbon atoms, for instance octyldodecanol, 2-butyloctanol, 2-hexyldecanol, 2-undecylpentadecanol or oleyl alcohol.

According to one embodiment, the composition according to the invention comprises at least one C₈-C₁₆ branched alkane, preferably chosen from isododecane and isohexadecane.

In the composition of the invention, all the hydrocarbon-based oils being considered together, the total amount of oil(s) may range, for example, from 15% to 60%, preferably from 20% to 55% by weight, better still from 25% to 50% by weight and even better still from 25% to 40% by weight relative to the total weight of the composition.

### Additional Surfactant

The two-phase composition may optionally comprise at least one surfactant other than alkylpolyglucosides in one or other of the phases. When it contains a surfactant, this surfactant may be of the anionic, nonionic or amphoteric type, but it is preferably of the nonionic and/or anionic type. It is preferably present in the hydrophilic phase.

The total amount of surfactant(s) as active material should be an amount such that the two phases remain separate at rest and do not mix to form an emulsion. This amount should generally be less than or equal to 2% by weight relative to the total weight of the composition. It may range, for example, from 0.01% to 1.5% by weight, preferably from 0.025% to 1% by weight and better still from 0.05% to 0.5% by weight relative to the total weight of the composition.

Among the nonionic surfactants, those that are particularly preferred are:

polyoxyethylenated fatty esters of sorbitol such as the product sold under the name Tween 20 by the company ICI,

polyoxyethylenated fatty alcohols such as the product sold under the name Remcopal 21912 AL by the company Gerland,

polyoxyethylenated alkylphenols such as the product sold under the name Triton X 100 by the company Röhm & Haas, and

condensates of ethylene oxide and propylene oxide such as those sold under the name Synperonic PE by the company ICI and in particular those referenced L 31, L 64, F 38, F 88, L 92, P 103, F 108 and F 127,

fatty acid esters of glycerol or of polyglycerol, for instance glyceryl isostearate, poly(3-glyceryl)diisostearate or glyceryl caprylate,

ethers of polyethylene glycol and/or polypropylene glycol and of glycerol, such as glycereth-7, glycereth-26 and PPG-24 glycereth-24,

esters derived from the reaction a) of fatty acids and b) of polyethylene glycol and/or polypropylene glycol glycerol ethers, for instance glycereth-2 cocoate or glycereth-25 PCA isostearate,

fatty acid esters of sucrose comprising from 12 to 30 carbon atoms are in particular 14 to 20 carbon atoms, the said esters is possibly comprising from 2 to 5 fatty chains, for instance sucrose distearate, sucrose tristearate or sucrose palmitate.

Among the anionic surfactants, mention may be made especially of:

alkyl sulfates, alkyl ether sulfates and salts thereof, especially the sodium salts thereof, for instance the mixture of sodium laureth sulfate/magnesium laureth sulfate/sodium laureth-8 sulfate/magnesium laureth-8 sulfate, sold under the name Texapon ASV by the company Henkel; sodium lauryl ether sulfate (70/30 C12-14) (2,2 OE) sold under the names Sipon AOS 225 or Texapon N702 Pate by the company Henkel, ammonium lauryl ether sulfate (70/30 C12-14) (3 OE) sold under the name Sipon LEA 370 by the company Henkel; the ammonium (C12-C14)alkyl ether (9 OE) sulfate sold under the name Rhodapex AB/20 by the company Rhodia Chimie;

alkyl sulfoacetates, such as the product sold under the name Lathanol LAL by the company Stepan;

alkyl sulfosuccinates, for example oxyethylenated (3 EO) lauryl alcohol monosulfosuccinate (70/30 C12/C14) sold under the names Setacin 103 Special and Rewopol SB-FA 30 K 4 by the company Witco, the disodium salt of a C12-C14 alcohol hemisulfosuccinate, sold under the name Setacin F Special Paste by the company Zschimmer Schwarz, the oxyethylenated (2 EO) disodium oleamidosulfosuccinate sold under the name Standapol SH 135 by the company Henkel, the oxyethylenated (5 EO) laurylamide monosulfosuccinate sold under the name Lebon A-5000 by the company Sanyo, the oxyethylenated (10 EO) disodium salt of lauryl citrate monosulfosuccinate sold under the name Rewopol SB CS 50 by the company Witco, and the disodium salt of ricinoleic acid monoethanolamide monosulfosuccinate sold under the name Rewoderm S 1333 by the company Witco;

polypeptides that are obtained, for example, by condensation of a fatty chain onto the amino acids of cereals and especially of wheat and oat, for instance the potassium salt of hydrolyzed lauroyl wheat protein, sold under the name Aminofoam W OR by the company Croda, the triethanolamine salt of hydrolyzed cocoyl soybean protein, sold under the name May-Tein SY by the company Maybrook, the sodium salt of lauroyl oat amino acids, sold under the name Proteol Oat by the company SEPPIC, the collagen hydrolysate grafted onto coconut fatty acid, sold under the name Geliderm 3000 by the company Deutsche Gelatine, and the soybean proteins acylated with hydrogenated coconut acids, sold under the name Proteol VS 22 by the company SEPPIC;

amino acid derivatives, for example among sarcosinates and especially acylsarcosinates such as the sodium lauroylsarcosinate sold under the name Sarkosyl NL 97 by the company Ciba or sold under the name Oramix L 30 by the company SEPPIC, sodium myristoyl sarcosinate, sold under the name Nikkol Sarcosinate MN by the company Nikkol, sodium palmitoyl sarcosinate, sold under the name Nikkol Sarcosinate PN by the company Nikkol; alaninates, such as the sodium N-lauroyl-N-methylamidopropionate sold under the name Sodium Nikkol Alaninate LN 30 by the company Nikkol or sold under the name Alanone ALE by the company Kawaken, and N-lauroyl-N-methylalanine triethanolamine, sold under the name Alanone ALTA by the company Kawaken; N-acylglutamates, such as the triethanolamine monococoylglutamate sold under the name Acylglutamate CT-12 by the company Ajinomoto, and the triethanolamine lauroylglutamate sold under the name Acylglutamate LT-12 by the company Ajinomoto; aspartates, such as the mixture of triethanolamine N-lauroylaspartate and triethanolamine N-myristoylaspartate, sold under the name Asparack LM-TS2 by the company Mitsubishi; glycine derivatives, such as sodium N-cocoylglycinate and potassium N-cocoylglycinate, such as the products sold under the names Amilite GCS-12 and Amilite GCK-12 by the company Ajinomoto;

sulfonates, for example, the α-olefinsulfonates, such as the sodium α-olefinsulfonate (C14-C16), sold under the name Bio-Terge AS-40 by the company Stepan, sold under the names Witconate AOS Protégé and Sulframine AOS PH 12 by the company Witco or sold under the name Bio-Terge AS-40 CG by the company Stepan, secondary sodium olefinsulfonate, sold under the name Hostapur SAS 30 by the company Clariant; or linear alkylarylsulfonates, such as the sodium xylenesulfonate, sold under the names Manrosol SXS30, Manrosol SXS40 and Manrosol SXS93 by the company Manro;

isethionates, especially acylisethionates, such as sodium cocoylisethionate, such as the product sold under the name Jordapon CI P by the company Jordan.

Among the amphoteric or zwitterionic surfactants, mention may be made especially of:

alkylamido alkylamine derivatives such as N-disodium N-cocoyl-N-carboxymethoxyethyl-N-carboxymethylethylenediamine (CTFA name: Disodium cocoamphodiacetate) sold as an aqueous saline solution under the name Miranol C2M Conc NP by the company Rhodia Chimie; N-sodium N-cocoyl-N-hydroxyethyl-N-carboxymethylethylenediamine (CTFA name: sodium cocamphoacetate) and the mixture of coconut acid ethanolamides (CTFA name: Cocamide DEA);

betaines, for instance cocoylbetaine, such as the product sold under the name Dehyton AB-30 by the company Henkel, laurylbetaine, such as the product sold under the name Genagen KB by the company Clariant, oxyethylenated (10 OE) laurylbetaine, such as the product sold under the name Lauryl Ether (10 OE) Betaine by the company Shin Nihon Rica, or oxyethylenated (10 OE) stearylbetaine, such as the product sold under the name Stearyl Ether (10 OE) Betaine by the company Shin Nihon Rica;

alkylamidopropylbetaines and derivatives thereof, for instance the cocamidopropylbetaine sold under the name Lebon 2000 HG by the company Sanyo, or sold under the name Empigen BB by the company Albright & Wilson, the lauramidopropylbetaine sold under the name Rewoteric AMB12P by the company Witco, such as cocamidopropylbetaine, for instance the product sold under the name Tego Betaine by the company Goldschmidt;

imidazoline derivatives such as the product sold under the name Chimexane HD by the company Chimex, and

mixtures thereof.

### Adjuvants

The composition according to the invention, i.e. the hydrophilic phase and/or the oily phase, may also contain conventional cosmetic adjuvants or additives, which will be in one or other phase depending on their hydrophilic or lipophilic nature, for instance hydrophilic gelling agents, preserving agents and bactericides, dyes, softeners, buffers, humectants, UV-screening agents (or sunscreens), electrolytes such as sodium chloride or a pH regulator, for example citric acid or sodium hydroxide, and mixtures thereof.

Depending on the desired viscosity of the composition according to the invention, it is possible to incorporate therein one or more hydrophilic gelling agents. Examples of hydrophilic gelling agents that may be mentioned include modified or unmodified carboxyvinyl polymers, such as the products sold under the name Carbopol (INCI name: carbomer) by the company Noveon; polyacrylamides; optionally crosslinked and/or neutralized 2-acrylamido-2-methylpropanesulfonic acid homopolymers and copolymers, such as the poly(2-acrylamido-2-methylpropanesulfonic acid) sold by the company Clariant under the name Hostacerin AMPS (INCI name: ammonium polyacryldimethyltauramide); polysaccharide biopolymers such as xanthan gum, guar gum, alginates and celluloses, which may or may not be modified; and mixtures thereof. When they are present, these gelling agents must be introduced in an amount such that they do not modify the properties of the composition according to the invention. Lipophilic gelling agents that may be mentioned include alkene copolymers, for instance block copolymers of “diblock”, “triblock” or “radial” type, of the polystyrene/polyisoprene or polystyrene/polybutadiene type, such as the products sold under the name Luvitol HSB® by the company BASF, of the polystyrene/copoly(ethylene-propylene) type, such as the products sold under the name Kraton® by the company Shell Chemical Co., or of the polystyrene/copoly(ethylene-butylene) type, and mixtures of triblock and radial (star) copolymers in isododecane, such as those sold by the company Penreco under the name Versagel®, for instance the mixture of butylene/ethylene/styrene triblock copolymer and of ethylene/propylene/styrene star copolymer in isododecane (Versagel M 5960).

The gelling agent may be present in an active material content ranging from 0.05% to 10% by weight and preferentially from 0.1% to 5% by weight relative to the total weight of the composition.

Preserving agents that may be used include any preserving agent usually used in the fields under consideration, for instance parabens and chlorhexidine gluconate.

An example of a bactericidal that may be used is a glyceryl mono(C₃-C₉)alkyl- or (C₃-C₉)alkenyl ether, the manufacture of which is described in the literature, in particular in E. Baer, H. O. L. Fischer—J. Biol. Chem. 140-397-1941. Among these glyceryl mono(C₃-C₉)alkyl- or (C₃-C₉)alkenyl ethers, use is preferably made of 3-[(2-ethylhexyl)oxy]-1,2-propanediol, 3-[(heptyl)oxy]-1,2-propanediol, 3-[(octyl)oxy]-1,2-propanediol and 3-[(allyl)oxy]-1,2-propanediol. A glyceryl mono(C₃-C₉)alkyl ether that is more particularly preferred according to the present invention is 3-[(2-ethylhexyl)oxy]-1,2-propanediol, sold by the company Schulke & Mayr G.m.b.H. under the trade name Sensiva SC 50 (INCI name: Ethylhexylglycerin).

Among the softeners, mention may be made in particular of allantoin and bisabolol, planktons, and certain plant extracts, such as rose extracts and melilot extracts.

According to the invention, the composition may also preferably comprise in the hydrophilic phase a dephasing agent in a proportion ranging, for example, from 0.025% to 5% by weight relative to the total weight of the composition.

Examples of dephasing agents that may be mentioned include alkyldimethylbenzylammonium chlorides as described in document EP-A-603 080, and especially benzalkonium chloride, and mixtures containing it; alkoxylated alkyl glucosides comprising a quaternary ammonium group and especially lauryl methyl gluceth-10 hydroxypropyldimonium chloride, as described in document EP-A-847 746; vinylpyrrolidone polymers and copolymers and especially the polyvinylpyrrolidone/hexadecene copolymer as described in document WO-A-99/56704; and mixtures thereof.

When such an agent is present, the ratio between the surfactant and the dephasing agent preferably ranges from 0.005/1 to 200/1 and better still from 0.01/1 to 120/1.

As active agents that may be used in the composition of the invention, i.e. in the hydrophilic phase and/or the oily phase, examples that may be mentioned include enzymes (for example lactoperoxidase, lipase, protease, phospholipase, cellulases); flavonoids; moisturizers, such as protein hydrolysates; sodium hyaluronate; anti-inflammatory agents; procyannidol oligomers; vitamins such as vitamin A (retinol), vitamin E (tocopherol), vitamin C (ascorbic acid), vitamin B5 (panthenol), vitamin B3 (niacinamide), derivatives of these vitamins (in particular esters) and mixtures thereof; urea; caffeine; depigmenting agents such as kojic acid, hydroquinone and caffeic acid; salicylic acid and derivatives thereof; α-hydroxy acids, such as lactic acid and glycolic acid and derivatives thereof; retinoids, such as carotenoids and vitamin A derivatives; hydrocortisone; melatonin; extracts of algae, of fungi, of plants, of yeasts, of bacteria; steroids; antibacterial active agents, such as 2,4,4′-trichloro-2′-hydroxydiphenyl ether (or triclosan), 3,4,4′-trichlorocarbanilide (or triclocarban) and the acids indicated above, and in particular salicylic acid and derivatives thereof; tensioning agents; ceramides; essential oils; and mixtures thereof and any active agent that is suitable for the final purpose of the composition.

The UV-screening agents that may be used in the composition of the invention are organic. They may be present in an active-material amount ranging from 0.01% to 20% by weight of active material, preferably from 0.1% to 15% by weight and better still 0.2% to 10% by weight relative to the total weight of the composition.

The compositions described above may be conditioned, in a known manner, in a bottle with a single compartment. The user must then shake the bottle before pouring its contents onto a pad of cotton wool. The product may also be conditioned in a bottle of “pump-action bottle” type. It may also be envisaged for the two phases of the composition to be introduced into two independent compartments of the same bottle, a system being provided for mixing them together at the time of dispensing. Such devices are described, for example, in documents EP-A-497 256 and FR-A-2 697 233.

The composition according to the invention may be used for any topical application, and may especially constitute a cosmetic or dermatological composition. It may in particular be used for caring for, cleansing and/or removing makeup from the skin, the lips and/or the eyes, and also as a haircare composition.

Thus, a subject of the invention is also the cosmetic use of a cosmetic composition as defined above, for caring for, removing makeup from and/or cleansing the skin, the lips and/or the eyes, and/or for haircare.

A subject of the invention is also a cosmetic process for caring for, removing makeup from and/or cleansing the skin, the lips and/or the eyes, characterized in that a cosmetic composition as defined above is applied to the skin, the lips and/or the eyes.

A subject of the present invention is also a cosmetic haircare process, characterized in that a cosmetic composition as defined above is applied to the hair.

### EXAMPLES

The examples below of compositions according to the invention are given as illustrations with no limiting nature. Unless otherwise mentioned, the amounts indicated are expressed as weight percentages.

The makeup-removing compositions presented below were prepared.

Procedure: The constituents of the oily phase, on the one hand, and those of the aqueous phase, on the other hand, are mixed. The two phases are then mixed together.

Example 1
Hydrophilic phase (70% by weight of the composition)

| Water | qs |
|---|---|
| (50/50 C8/C10) Alkyl polyglucoside as an | 0.250% |
| aqueous 60% solution - Oramix CG 110 | |
| from SEPPIC | |
| Hydrophilic active agents | 2.000% |
| Preserving agents | 0.100% |

Lipophilic phase (30% by weight of the composition)

| | |
|---|---|
| Isopropyl palmitate | 39.999% |
| Isocetyl stearate | 60.000% |
| Lipophilic dye | 0.001% |

Example 2
Hydrophilic phase (70% by weight of the composition)

| Water | qs |
|---|---|
| (50/50 C8/C10) Alkyl polyglucoside as an | 0.250% |
| aqueous 60% solution - Oramix CG 110 | |
| from SEPPIC | |
| Hydrophilic active agents | 1.470% |
| Preserving agents | 0.100% |

Lipophilic phase (30% by weight of the composition)

| | |
|---|---|
| Isopropyl palmitate | 39.999% |
| Isohexadecane | 50.000% |
| Isocetyl stearate | 10.000% |
| Lipophilic dye | 0.001% |

Example 3
Hydrophilic phase (70% by weight of the composition)

| Water | qs |
|---|---|
| (50/50 C8/C10) Alkyl polyglucoside as an | 0.250% |
| aqueous 60% solution - Oramix CG 110 | |
| from SEPPIC | |
| Hydrophilic active agents | 2.000% |
| Preserving agents | 1.000% |

Lipophilic phase (30% by weight of the composition)

| | |
|---|---|
| Ethylhexyl palmitate | 99.999% |
| Lipophilic dye | 0.001% |

Example 4
Hydrophilic phase (70% by weight of the composition)

| Water | qs |
|---|---|
| (50/50 C8/C10) Alkyl polyglucoside as an | 0.250% |
| aqueous 60% solution - Oramix CG 110 | |
| from SEPPIC | |
| Hydrophilic active agents | 2.000% |
| Preserving agents | 1.000% |

Lipophilic phase (30% by weight of the composition)

| | |
|---|---|
| Isopropyl myristate | 99.999% |
| Lipophilic dye | 0.001% |

Example 5
Hydrophilic phase (70% by weight of the composition)

| Water | qs |
|---|---|
| (50/50 C8/C10) Alkyl polyglucoside as an | 0.250% |
| aqueous 60% solution - Oramix CG 110 | |
| from SEPPIC | |
| Hydrophilic active agents | 1.000% |
| Preserving agents | 1.000% |

Lipophilic phase (30% by weight of the composition)

| | |
|---|---|
| Dicaprylyl carbonate | 99.999% |
| Lipophilic dye | 0.001% |

Example 6
Hydrophilic phase (70% by weight of the composition)

| Water | qs |
|---|---|
| Decyl glucoside as an aqueous 40% | 0.250% |
| solution - Mydol 10 from Kao | |
| Hydrophilic active agents | 2.000% |
| Preserving agents | 0.100% |

Lipophilic phase (30% by weight of the composition)

| | |
|---|---|
| Isopropyl palmitate | 39.999% |
| Isocetyl stearate | 60.000% |
| Lipophilic dye | 0.001% |

Compositions are obtained, which, when at rest, comprise an aqueous phase and a separate oily phase, which are coloured and transparent. On shaking, the two phases give a transparent composition, having a sharp interface and a mixing time allowing good removal of makeup from the skin, the lips and the eyes.

1. Composition for topical application, consisting of a hydrophilic phase and of a separate oily phase,
the hydrophilic phase comprising from 0.05% to 1.5% by weight of at least one non-ethoxylated alkylpolyglucoside relative to the total weight of the hydrophilic phase, and
the oily phase comprising at least 10% by weight, relative to the total weight of the oily phase, of at least one ester with a melting point of less than 10° C. chosen from fatty acid esters with a chain length ranging from 8 to 18 carbon atoms and fatty alcohol esters with a chain length ranging from 8 to 18 carbon atoms, and less than 10% by weight, relative to the total weight of the oily phase, of volatile silicone oil(s),
with the exclusion of compositions Comprising 75% by weight of hydrophilic phase and 25% by weight of lipophilic phase relative to the weight of the composition, the lipophilic phase containing 45% by weight of isopropyl myristate, relative to the weight of the lipophilic phase, and the hydrophilic phase comprising 0.1% by weight, relative to the weight of the hydrophilic phase, of (85/10/5 C10/12/14)alkylpolyglucoside (1,4) as an aqueous solution containing 55% active material or of (50/50 C8/C10)alkylpolygiticoside as an aqueous solution containing 60% active material.

2. Composition according to claim 1, in which the weight ratio between the hydrophilic phase and the oily phase ranges from 90/10 to 10/90.

3. Composition according to claim 1, wherein the alkylpolyglycoside corresponds to the following structure:
R(O)(G)ₓ
in which the radical R is a linear or branched C₆-C₃₀ alkyl radical, G is a saccharide residue and x ranges from 1 to 5.

4. Composition according to claim 1, in which the alkylpolyglucoside contains an alkyl group comprising from 6 to 30 carbon atoms and containing a glucoside group.

5. Composition according to claim 1, in which the alkylpolyglycoside is chosen from decylglucoside and caprylyl/capryl glucoside.

6. Composition according to claim 1, wherein the alkylpolyglycoside may be used as a mixture with at least one fatty alcohol.

7. Composition according to claim 6, in which the fatty alcohol/alkylpolyglycoside mixture is chosen from:
cetylstearyl alcohol/cocoyl glucoside,
arachidyl alcohol and behenyl alcohol/arachidyl glucoside,
myristyl alcohol/myristyl glucoside,
cetylstearyl alcohol/cetylstearyl glucoside,
C₁₄-C₂₂ alcohol/C₁₂-C₂₀ alkyl glucoside,
cocoyl alcohol/cocoyl glucoside,
isostearyl alcohol/isostearyl glucoside,
cetylstearyl alcohol/cetylstearyl glucoside.

8. Composition according to claim 1, wherein the hydrophilic phase comprises from 0.05% to 1.5% by weight of at least one non-ethoxylated alkylpolyglucoside relative to the total weight of the hydrophilic phase.

9. Composition according to claim 1, wherein the oily phase comprises less than 5% by weight of volatile silicone oils relative to the total weight of the oily phase.

10. Composition according to claim 1, in which the volatile silicone oil is chosen from cyclopentasiloxane and cyclohexasiloxane.

11. Composition according to claim 1, in which the ester with a melting point of less than 10° C. may be chosen from the oils of formula R1COOR2 in which:
R1 represents a substituted or unsubstituted, linear or branched alkyl or alkoxy group comprising from 8 to 18 carbon atoms, and possibly comprising one or more ethylenic bonds, and
R2 represents a substituted or unsubstituted, linear or branched alkyl radical comprising from 2 to 20 carbon atoms and possibly comprising one or more ethylenic bonds.

12. Composition according to claim 1, in which the ester with a melting point, of less than 10° C. is chosen from: isocetyl stearate, isopropyl myristate, dicaprylyl carbonate, ethyl hexyl palmitate.

13. Composition according to claim 1, wherein isopropyl palmitate is also comprised.

14. Composition according to claim 1, wherein at least one branched C8-C16 alkane is also compromised.

15. Cosmetic process for removing makeup from, for cleansing and/or for caring for the skin, the lips and/or the eyes or the hair, wherein a cosmetic composition according to claim 1 is applied to the skin, the lips and/or the eyes.
